(19) United States
(12) Patent Application Publication
(10) Pub. No.: US 2023/0405201 A1
(43) Pub. Date: Dec. 21, 2023

(54) TECHNIQUES FOR INCREASING RED BLOOD CELL COUNT

(71) Applicants: FRESENIUS MEDICAL CARE HOLDINGS, INC., Waltham, MA (US); FRESENIUS MEDICAL CARE DEUTSCHLAND GMBH, Bad Homburg (DE)

(72) Inventors: Peter Kotanko, New York, NY (US); Nadja Grobe, Huntington, NY (US); Christoph Zaba, Krems (AT); David Joerg, Bad Homburg (DE); Frank Gebauer, Krems (AT)

(21) Appl. No.: 18/032,931

(22) PCT Filed: Nov. 12, 2021
(86) PCT No.: PCT/US2021/059127

§ 371 Date: Apr. 20, 2023

Related U.S. Application Data
(60) Provisional application No. 63/113,403, filed on Nov. 13, 2020.

(51) Int. Cl. A61M 1/34

(52) U.S. Cl. CPC A61M 1/3486; A61M 2202/0498

(57) ABSTRACT

The described technology may include treatment processes to increase the red blood cell (RBC) population of individuals, particularly chronic kidney disease (CKD) patients with renal anemia, by reducing an amount of Piezo1 chemical agonists in the blood of patients. In one embodiment, a method of treating a patient with renal anemia may include increasing RBC lifespan of an RBC population of the patient via reduction of a Piezo1 channel activation duration of at least a portion of the RBC population by reducing an amount of a target uremic compound in the blood of the patient, the target uremic compound having a form that prolongs the Piezo1 channel activation duration, wherein the amount of the target uremic compound may be reduced via selectively removing at least a portion of the target uremic compound from the blood of the patient. Other embodiments are described.

### CROSS-REFERENCE TO RELATED APPLICATION

This application claims the benefit of the filing date of U.S. Provisional Patent Application No. 63/113,403, filed Nov. 13, 2020 and titled “Methods and Apparatuses for Increasing Red Blood Cell Lifespan,” the contents of which are incorporated by reference herein in their entirety.

### FIELD

The disclosure generally relates to processes for increasing the lifespan of red blood cells in the human circulatory system and, more particularly, to techniques for increasing and/or maintaining a healthy red blood cell count through the increase of red blood cell lifespan via affecting red blood cell ion channel activation.

### BACKGROUND

In healthy individuals, red blood cells (RBC) have an approximate lifespan of 100 to 120 days in circulation. For patients with kidney disease (for instance, chronic kidney disease (CKD) patients requiring dialysis treatment), RBC lifespan is often reduced, compounding renal anemia, a complication associated with reduced quality of life and increased morbidity and mortality.

A standard treatment for anemia may include a drug regimen to increase RBC population or count and/or to increase RBC lifespan. Example drug regimens may include administration of erythropoietin (EPO), EPO stimulating agents (ESA), and/or iron supplements. The exact biological mechanisms that determine normal RBC life span in healthy individuals and reduced RBC lifespan in anemia patients are not completely understood. Determining and addressing these mechanisms behind reduced RBC lifespan in dialysis patients would be beneficial to anemic patients as well as healthcare providers. For instance, increasing RBC lifespan in patients may improve patient quality of life, reduce health issues and morbidity, and decrease costs and reliance on medication (for example, if the administration of EPO could be reduced or even eliminated).

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In one embodiment, a method of treating a patient with renal anemia may include increasing a red blood cell (RBC) lifespan of an RBC population of the patient via reduction of a Piezo1 channel activation duration of at least a portion of the RBC population by reducing an amount of a target uremic compound in the blood of the patient, the target uremic compound having a form that prolongs the Piezo1 channel activation duration, wherein the amount of the target uremic compound may be reduced via selectively removing at least a portion of the target uremic compound from the blood of the patient.

In some embodiments of the method, the target uremic compound may be 3-carboxy-4-methyl-5-propyl-2-furanpropionate (CMPF). In various embodiments of the method, the method may include monitoring an average RBC lifespan of the using a mathematical model of erythropoiesis. In some embodiments of the method, the patient may be receiving a dosage (for example, a maximum dosage) of at least one erythropoietin stimulating agents (ESA) to treat renal anemia. In various embodiments, the patient may be receiving a dosage of Hypoxia-inducible factor (HIF) prolyl hydroxylase (PH or PHD) enzyme inhibitors, such as Roxadustat, to treat renal anemia. In various embodiments of the method, the method may include reducing the ESA (and/or HIF-PHD) dosage based on an increase in the RBC lifespan of the patient.

In exemplary embodiments of the method, selectively removing the target uremic compound includes an adsorption process performed on blood of the patient. In some embodiments of the method, the adsorption process comprises fractionated plasma separation and adsorption (FPSA). In various embodiments of the method, the adsorption process using a ligand to adsorb CMPF, the ligand having a binding affinity to CMPF in the range of about K₁=10⁶ to 10⁸. In some embodiments of the method, the method may include performing apheresis to selectively remove the target uremic compound. In various embodiments of the method, the method may include performing apheresis with a displacer targeting an RBC binding site of at least one uremic compound. In exemplary embodiments of the method, the displacer may include dithymoquinone (DTQ) or chemical analogues thereof.

In one embodiment, an apparatus for treating a patient with renal anemia may include a target compound reduction system configured to engage blood of the patient to reduce an amount of a target compound from the blood of the patient by selectively removing at least a portion of the target compound from the blood of the patient, wherein reducing the amount of the target compound in the blood of the patient increases a red blood cell (RBC) lifespan of an RBC population in the blood of the patient via reduction of a Piezo1 channel activation duration of at least a portion of the RBC population, the target compound having a form that prolongs the Piezo1 channel activation duration.

In some embodiments of the apparatus, the target compound may be a uremic compound. In various embodiments of the apparatus, the target compound may be 3-carboxy-4-methyl-5-propyl-2-furanpropionate (CMPF). In exemplary embodiments of the apparatus, the target compound reduction system operative to perform an adsorption process on the blood of the patient. In various embodiments of the apparatus, the adsorption process comprises fractionated plasma separation and adsorption (FPSA). In some embodiments of the apparatus, the adsorption process uses a ligand to adsorb CMPF, the ligand having a binding affinity to CMPF in the range of about K₁=10⁶ to 10⁸. In various embodiments of the apparatus, the target compound reduction system may be operative to perform apheresis to selectively remove the target compound. In exemplary embodiments of the apparatus, apheresis may be performed with a displacer targeting a RBC binding site of the at least one target compound. In various embodiments of the apparatus, the displacer may include dithymoquinone (DTQ) or chemical analogues thereof.

### BRIEF DESCRIPTION OF THE DRAWINGS

By way of example, specific embodiments will now be described, with reference to the accompanying drawings, in which:

FIG. 1 illustrates exemplary ion channel activation of a red blood cell (RBC) in accordance with the present disclosure;

FIG. 2 illustrates exemplary information associated with Piezo1 activation and RBC lifespan in accordance with the present disclosure;

FIG. 3A illustrates exemplary Piezo1 agonists in accordance with the present disclosure;

FIG. 3B illustrates exemplary Piezo1 agonist activation sites in a RBC in accordance with the present disclosure;

FIG. 4 illustrates carboxy-4-methyl-5-propyl-2-furanpropionate (CMPF) in accordance with the present disclosure;

FIG. 5A illustrates exemplary pathways that lead to decreased RBC population in accordance with the present disclosure;

FIG. 5B illustrates exemplary pathways for achieving a healthy RBC population range using treatment processes in accordance with the present disclosure;

FIG. 6 illustrates a first exemplary Piezo1 agonist removal system in accordance with the present disclosure;

FIG. 7A illustrates a second exemplary Piezo1 agonist removal system in accordance with the present disclosure; and

FIG. 7B illustrates a second exemplary Piezo1 agonist removal system in accordance with the present disclosure.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and convey the scope of the subject matter to those skilled in the art. In the drawings, like numbers refer to like elements throughout.

The embodiments described in the present disclosure may generally be directed toward treatment processes, methods, systems, and/or apparatuses for increasing the red blood cell (RBC) count in individuals. In one example, the treatment processes described in the present disclosure may be used for the treatment of individuals with low RBC count, including, in particular, patients with anemia. Low RBC count may be due to one or both of reduced RBC production (for example, due to deficiencies of erythropoietin, iron, vitamins, trace elements, and/or the like) and/or reduced RBC lifespan (for example, due to blood loss, eryptosis, hemolysis, and/or the like. Therefore, treatment processes according to some embodiments may operate to increase RBC count by affecting erythropoiesis (RBC production) and/or eryptosis (RBC death).

Various embodiments may be beneficial for increasing RBC count in patients with chronic kidney disease (CKD) (i.e., that typically exhibit decreased RBC lifespan), including, in particular, patients with renal anemia. Globally, about 700 million patients suffer from chronic kidney disease (CKD). Most patients with advanced CKD stages develop renal anemia at some point, a complication associated with reduced quality of life and increased morbidity and mortality. Erythropoietin (EPO), the main erythropoiesis-stimulating hormone, is produced primarily by the kidneys. In CKD, EPO deficiency is frequent and a well-documented cause of renal anemia. Other contributing factors are absolute and/or functional iron deficiency, inflammation with increased hepcidin levels, and shortened RBC life span. Augmenting erythropoiesis is a widely applied treatment strategy, so that renal anemia is usually managed with erythropoiesis stimulating agents (ESAs; similar in effect to endogenous EPO), iron supplements, and, more recently, hypoxia-inducible factor prolyl hydroxylase inhibitors (HIF-PHD; for example, Roxadustat), drugs that increase EPO production, improve iron availability, and reduce hepcidin levels.

In healthy individuals, RBC life span is around 100 to 120 days. Shortened RBC life span is observed in most patients with advanced CKD and can contribute to the development of renal anemia. For example, in CKD patients on hemodialysis, average RBC life span is shortened to around 50 to 70 days. Since the steady-state number of circulating RBCs depends on the balance between RBC formation and RBC death, a shortened RBC lifespan is considered a major contributor to renal anemia. This suggests that interventions which systematically increase RBC lifespan in CKD patients may alleviate anemia, leading to a larger steady-state RBC pool and thus, higher blood hemoglobin concentrations. In addition, such interventions may reduce the overall number of ESAs needed to maintain adequate hemoglobin levels.

In some embodiments, treatment processes may be directed toward affecting the activation of ion channels embedded in the membrane of a RBC. In some embodiments, the ion channel may be an ion channel that has a role in regulating calcium (Ca, Ca++, Ca²⁺, intracellular Ca (iCa++), and/or the like) intake into RBCs. For example, the influx of Ca++ has been demonstrated to be a pivotal event in eryptosis (for example, when a Ca++ imbalance between Ca++ influx and efflux is created) (see, for example, Dias et al., “The Role of Eryptosis in the Pathogenesis of Renal Anemia: Insights from Basic Research and Mathematical Modeling,” Frontiers in Cell and Developmental Biology, 9 Dec. 2020, which is incorporated by reference as if fully set forth herein).

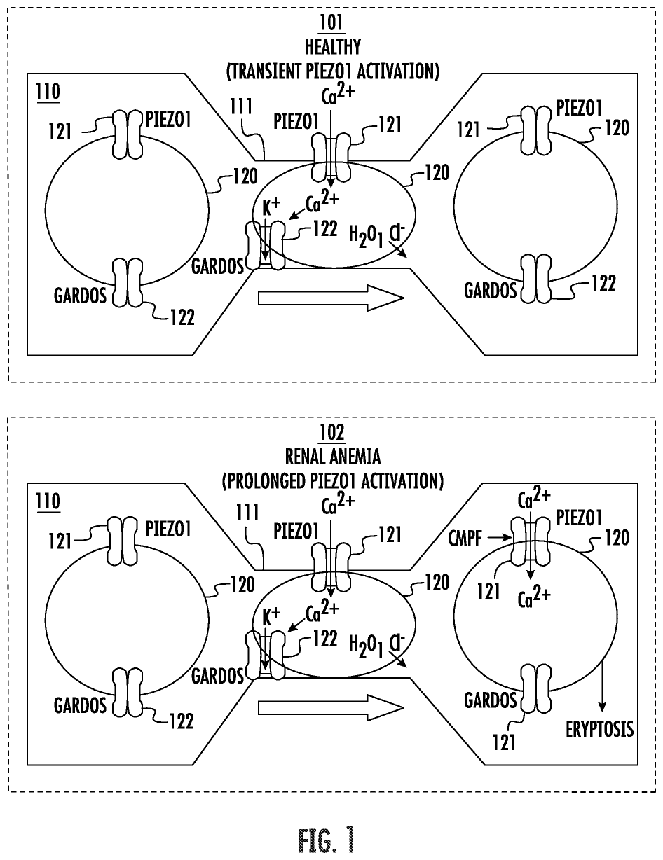

In various embodiments, the ion channel may be a Piezo channel that opens in response to a mechanical force applied to the RBC that allows Ca++ and/or other ions to enter the RBC (see, for example, FIG. 1). A non-limiting example of an ion channel may be the Piezo1 channel that operates to regulate, among other things, RBC cellular volume.

In exemplary embodiments, a treatment process may operate to reduce Piezo1 channel activation in RBCs. In some embodiments, treatment processes may be directed toward removing, de-activating, or otherwise affecting Piezo1 channel agonists to reduce agonist-induced Piezo1 channel activation. Reducing Piezo1 channel activation may include reducing the number of activations and/or reducing the duration of channel activation. More specifically, some embodiments may include methods directed toward reducing or even eliminating non-mechanical based Piezo1 channel activation (number of activations and/or activation duration) caused by chemical activation, for instance, via Piezo1 channel agonists. One non-limiting example of a Piezo1 channel agonists may include a uremic compound. Another non-limiting example of a Piezo1 channel agonists may include 3-carboxy-4-methyl-5-propyl-2-furanpropionate (CMPF). In some embodiments, a system, device, and/or apparatus may be operative to perform the treatment processes described in the present disclosure for increasing RBC lifespan.

In general, Piezo1 channel activation of RBCs may affect RBC lifespan (i.e., the greater the amount of Piezo1 channel activation, the lower the RBC lifespan). Piezo1 channel activation may occur due to mechanical activation when an RBC flows through a portion of a blood vessel, spleen slit, or other structure that is smaller in diameter than the RBC (i.e., allowing the RBC to become more flexible to fit through the smaller diameter portion). However, Piezo1 channel activation may be prolonged via certain chemical compounds. Reduction of the chemical stimulation may reduce the overall Piezo1 channel activations of a RBC, thereby increasing RBC lifespan.

RBC lifespan scales allometrically with body mass across mammals, so that RBC in mammals with a low body mass have in most cases a shorter RBC life span compared to mammals with a higher mass. Across 4 orders of body mass, the number of circulations during RBC lifetime is remarkably constant (150,000 to 250,000 circulations) despite markedly different RBC lifespans. One biological reason for this phenomenon may be or may include a form of cumulative RBC “erosion,” for example, with each circulation, an RBC (measuring around 7 μm in diameter) needs to traverse capillaries with a much smaller diameter (2-5 μm). Accordingly, the RBC undergo geometrical changes and reduce their diameter by stretching, showing a remarkable ability to deform. The passage through a capillary may last about 700 msec.

Treatment processes according to some embodiments may provide multiple technological advantages over existing systems and methods. In one non-limiting technological advantage, treatment processes according to some embodiments are capable of treating anemia (for instance, achieving a healthy RBC count) without the use of drugs or with a reduced amount of drugs for the patient, which reduces costs and health impacts on the patient. In another non-limiting technological advantage, treatment processes according to some embodiments are capable of treating abnormal RBC count via removal of a Piezo1 agonist, which is not available using conventional treatment methods. In an additional, non-limiting technological advantage, treatment processes according to some embodiments may provide interventions which lower CMPF levels to systematically increase RBC lifespan in CKD patients, alleviate renal anemia, and reduce ESA needs.

FIG. 1 illustrates exemplary ion channel activation of a red blood cell (RBC) in accordance with the present disclosure. Illustrative examples of RBC deformation mechanisms are presented in Danielczok et al., “Red Blood Cell Passage of Small Capillaries Is Associated with Transient Ca²⁺-mediated Adaptations,” Frontiers in Physiology, Dec. 5, 2017, which is incorporated by reference as if fully set forth herein.

Panel 101 depicts transient Piezo1 activation in normal or healthy individuals. A shown in FIG. 1, a RBC 120 may be passing through a blood vessel, spleen slit, or other portion of human anatomy 110. RBC 120 may have a volume, diameter, circumference or other characteristic that is larger than a narrow portion 111 of blood vessel 110. Accordingly, RBC 120 cannot fit through narrow portion 111 without a change in shape.

RBC 120 deformation is mediated through a Piezo1 mechanosensitive ion channel 121. Mechanical stimulation (for example, an outer portion of RBC 120 being forced against the inner walls of blood vessel 110 particularly adjacent and/or within narrow portion 111) promotes Ca²⁺ influx via Piezo1 channel 121. Upon activation, Piezo1 121 opens a Ca²⁺ channel and Ca²⁺ flows along an electro-chemical gradient into RBC 120 where it activates a series of intracellular processes that result in higher RBC flexibility. For example, complexation of Ca²⁺ with calmodulin also occurs, which then collectively binds RBC-NOS. When RBC-NOS is activated, NO is produced, which binds to α- and β-spectrins, leading to increased flexibility and improved cellular deformability of RBC 120. In addition, RBC experiences a loss of water, Cl⁻, and K⁺, resulting in a decrease in RBC 120 volume. Accordingly, RBC 120 may move through narrow portion 111.

Activation of a Gardos channel 122 occurs in response to sustained Ca²⁺-influx. Gardos channel 122 opens to facilitate export of K⁺, which leads to a loss of intracellular fluid. Simultaneously, Ca²⁺ is transported out of RBC 120 via the plasma membrane Ca²⁺-ATPase (PMCA). Cell shrinkage of RBC 120 occurs and a temporary loss of deformability is also experienced by RBC 120. At this stage, RBC 120 has traveled through narrow portion 111 and Piezo1 channel 121 and Gardos channel 122 have closed.

While the Ca²⁺ influx is vitally important to the trans-capillary passage of RBC 120, the divalent cations need to swiftly efflux from RBC 120, because extended periods of increased intracellular Ca²⁺ levels stimulate processes that promote the destruction of RBC 120. Likewise, it is important that Piezo1 121 is activated for only a brief period, namely during RBC 120 passage through narrow portion 111 (for instance, a capillary of a larger blood vessel system). However, as shown in panel 102, patients with CKD, particularly those with renal anemia, may experience prolonged Piezo1 121 activation. In particular, Piezo1 121 may remain open in panel 102 after RBC 120 has traveled through narrow portion 111.

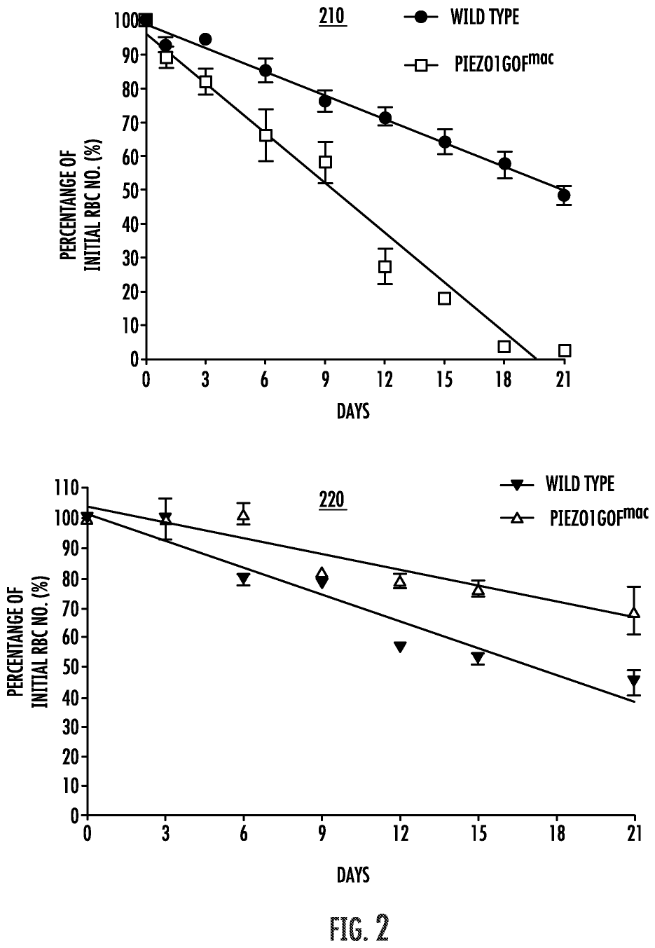

Sustained activation of Piezo1 results in a shortened RBC life span, as indicated by several mutations (e.g., R2456H, T2127M and E2496ELE) that exhibit a partial gain-of-function (GOF) phenotype and/or loss-of-function (LOF) phenotype. FIG. 2 illustrates exemplary information associated with Piezo1 activation and RBC lifespan in accordance with the present disclosure. More specifically, FIG. 2 depicts decreased RBC lifespan with Piezo1 GOF in graph 210 and increased RBC lifespan with Piezo1 LOF in graph 220. Accordingly, Piezo1 GOF mutations shorten RBC lifespan and Piezo1 LOF mutations increase RBC lifespan. Examples of Piezo1 GOF and LOF mutations and their effect on RBC lifespan may be found in Rotordam et al., “A novel gain-of-function mutation of Piezo1 is functionally affirmed in red blood cells by high-throughput patch clamp,” Haematologica, 104(5): e179-e183 (2019) and Ma et al., “Correlation between Inflammatory Biomarkers and Red Blood Cell Life Span in Chronic Hemodialysis Patients,” Blood Purification 2017; 43(1-3): 200-5, both of which are incorporated by reference as if fully set forth herein.

Accordingly, it appears that prolonged Piezo1 activation (for instance, via delayed Piezo1 channel activation) negatively affects RBC lifespan and, therefore, RBC count. In general, Piezo1, a mechanoreceptor located on the RBC surface, stimulates Ca²⁺ influx. This facilitates the passage of RBC through capillaries, spleen slits, and other narrow vessel structures. However, elevated Ca²⁺ is key trigger of eryptosis. Prolonged Piezo1 activation results in extended Ca²⁺ influx and excessive eryptosis. In addition, Piezo1 activation decelerates erythropoiesis and promotes cardiac hypertrophy.

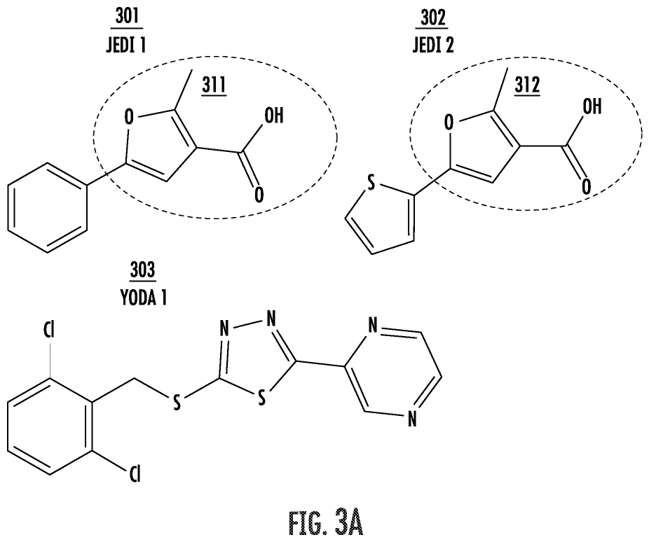

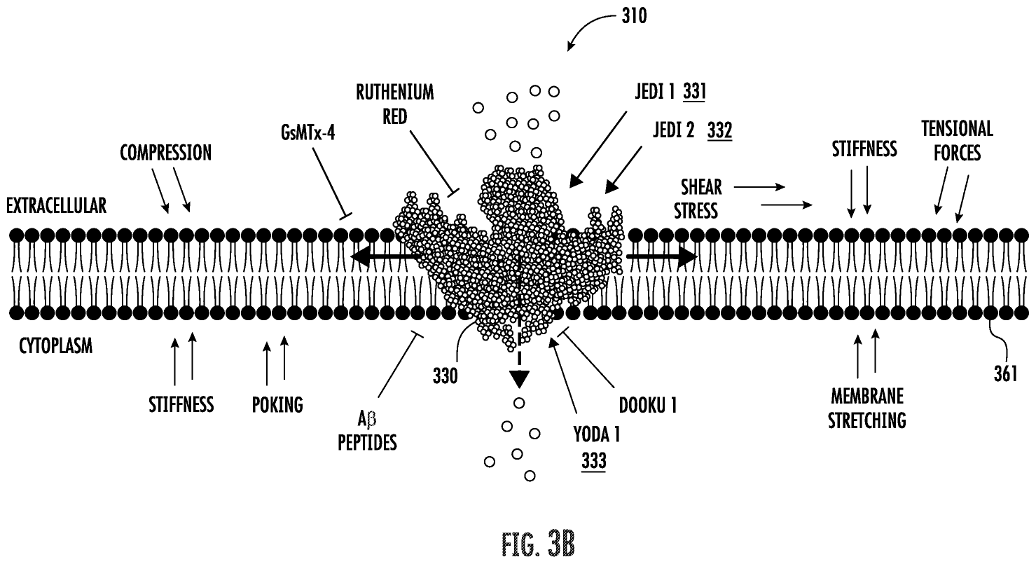

FIG. 3A illustrates exemplary Piezo1 agonists in accordance with the present disclosure. More specifically FIG. 3A depicts Piezo1 agonists Jedi1 301, Jedi2 302, and Yoda1 303. Small molecules 301-303 have been demonstrated to stimulate Piezo1 (see, for example, Wang et al., “A lever-like transduction pathway for long-distance chemical- and mechano-gating of the mechanosensitive Piezo1 channel,” Nature Communications, 9(1), Article Number 1300 (2018), the contents of which are incorporated by reference as if fully set forth herein). FIG. 3B illustrates exemplary Piezo1 agonist activation of a RBC in accordance with the present disclosure. As shown in FIG. 3B, a Piezo1 channel 330 may be embedded in a membrane 361 of a RBC 310. Piezo1 channel 330 may have activation sites for Jedi1 331, Jedi2 332, and Yoda1 333.

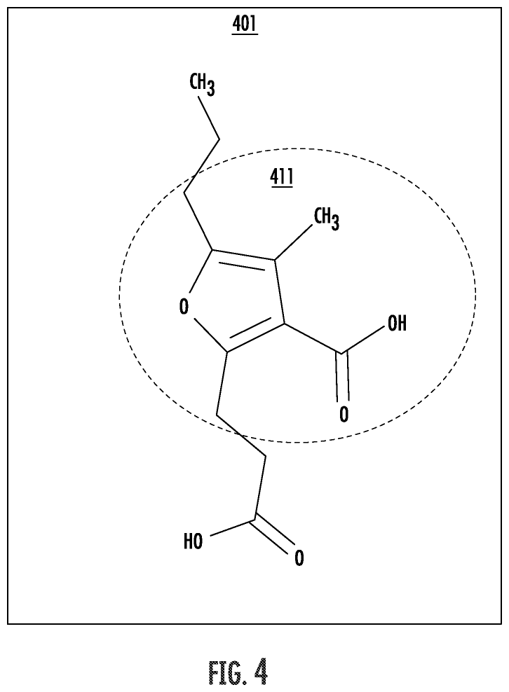

FIG. 4 illustrates carboxy-4-methyl-5-propyl-2-furanpropionate (CMPF) 401 in accordance with the present disclosure. CMPF is a 240 Da metabolite of furan fatty acids and a protein-bound uremic retention solute normally cleared by the healthy kidney. In CKD patients, CMPF levels are increased 5- to 15-fold compared to healthy subjects.

Referring back to FIG. 3A, group 311 of Jedi1 301 and group 312 of Jedi 302 are the moieties or “active centers” of the small molecules that activate Piezo1. Group or moiety 411 of CMPF 401 shares structural similarities with group 311 of Jedi1 301 and group 312 of Jedi2 302. Therefore, it appears that CMPF 401, for instance via moiety 411 as an “active center,” may activate Piezo1 in the same or similar manner as moieties 311 and 312 of Jedi1 301 and/or Jedi2 302, respectively. Accordingly, since CKD patients have elevated levels of CMPF, CKD patients therefore may have elevated Piezo1 activation through activation of Piezo1 via CMPF acting as a Piezo1 agonist.

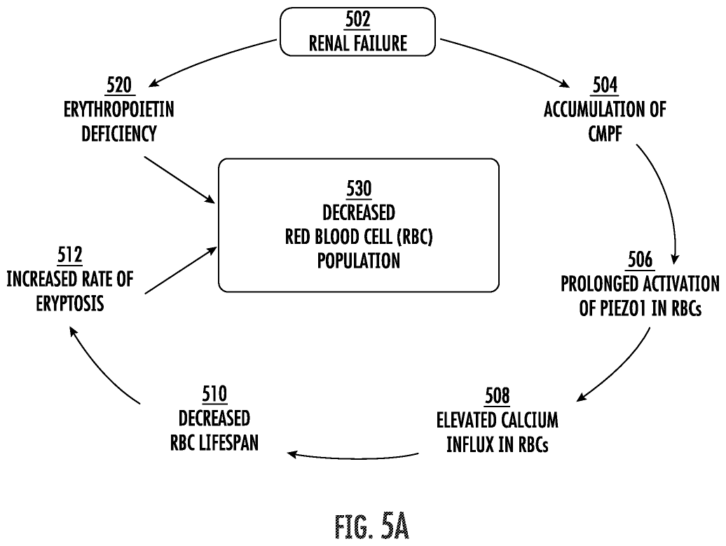

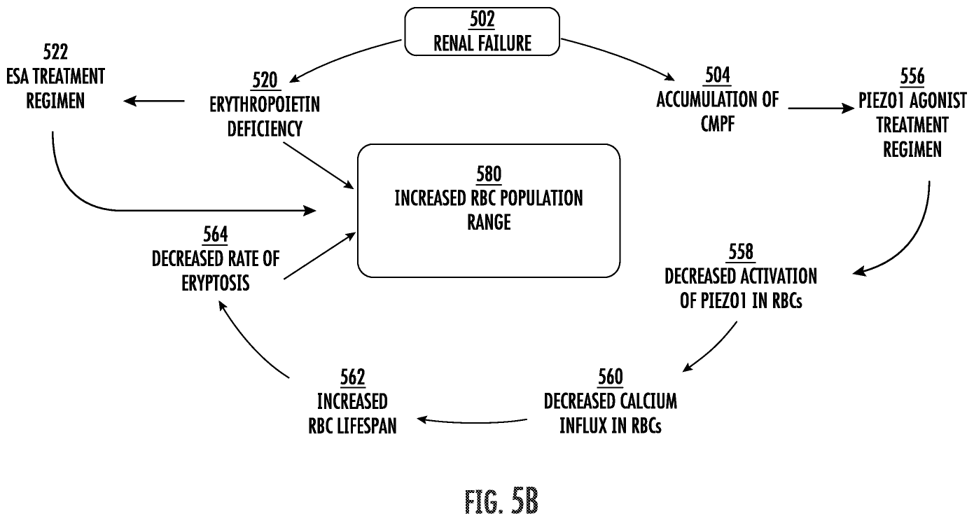

FIG. 5A illustrates exemplary pathways that lead to decreased RBC population in accordance with the present disclosure. As shown in FIG. 5A, renal failure in CKD patients 502 may lead to the well-established role of erythropoietin deficiency 520 in the pathogenesis of renal anemia that leads to decreased RBC population 530. In addition, renal failure 502 may also lead to increased CMPF levels and the accumulation of CMPF 504. CMPF may activate Piezo1. Therefore, increased CMPF levels may lead to prolonged activation of Piezo1 in RBCs 506 and, as a result, an elevated calcium influx in RBCs 508. The accumulation of CMPF in CKD patients may lead to decreased RBC lifespan 510 and an increased rate of eryptosis 512 which, ultimately, contribute to a decreased RBC population 530 and renal anemia.

Accordingly, some embodiments may include a treatment process directed toward CMPF levels because: (a) CMPF extends Piezo1 activation and calcium influx that triggers eryptotic pathways; (b) elevated CMPF levels in CKD reduce RBC lifespan and thus contribute to renal anemia; and (c) lowering CMPF levels in CKD patients will improve anemia by decreasing chemical activation of Piezo1.

FIG. 5B illustrates exemplary pathways for achieving a healthy or healthier RBC population range using treatment processes in accordance with the present disclosure. As shown in FIG. 5B, renal failure in CKD patients 502 may cause erythropoietin deficiency 520 and the accumulation of CMPF 504 that may each lead to a decreased RBC population (see, for example, FIG. 5A). An ESA treatment regimen 522 may lead to an increased RBC population range 580 (for instance, in comparison to no treatment).

In some embodiments, a treatment process may include a Piezo1 agonist treatment regimen 556. For example, a treatment process may include the removal or deactivation of one or more Piezo1 agonists, such as CMPF, which is increased in CKD patients. Removal of one or more Piezo1 agonists may lead to decreased activation of Piezo1 in RBCs 558 and, therefore, decreased calcium influx in RBCs 560 (for instance, in comparison to no treatment). In this manner, treatment processes may cause increased RBC lifespan 562 and a decreased rate of eryptosis 564, which may cause an increase in an RBC population range (for instance, in comparison to no treatment).

Although CMPF is used as an example in the present disclosure, embodiments are not so limited, for example, there exist many compounds (including uremic retention solutes) that may also interact with Piezo1 and thus affect RBC lifespan that may be treated using treatment processes (including, without limitation, Piezo1 agonist treatment regimens).

CMPF is a major endogenous ligand found in the serum of renal failure patients. CMPF may exist in a free form and/or a bound form. For example, CMPF may be bound to human serum albumin (HSA). In some embodiments, a treatment process may include an adsorptive-based removal of CMPF (and/or other Piezo1 agonists). For example, in an adsorptive removal of agonist(s), recognition of the location of the agonist's binding site/pocket to HSA, may be applied to design, develop, select or otherwise determine adsorptive materials removing specifically CMPF and/or other agonist(s) of interest. Further, knowing normal and elevated serum levels provides information on toxin mass to be depleted or removed. Accordingly, in some embodiments, treatment processes may be based on, inter alia, agonist binding site/pocket information and/or normal and elevated serum levels (toxin mass) for removing the targeted compound(s).

For example, for a CMPF and HSA method, CMPF is a typical representative of urofuranoid acids, which possess pronounced lipophilic properties and a high (for instance, at or about 10⁸ M⁻¹) constant of association with HSA molecules. CMPF possesses atrophy towards bilirubin's binding center on albumin molecules, also known as binding site 1. An average normal concentration for CMPF may be a mean (±SD) of about 4.6±1.8 with a range between about 3.6 and 7.7 mg/L. In uremic patients the mean concentration (CU) is about 25.9±10.2 mg/L (with a range of about 3.7 to 94 mg/L).

Accordingly, the relative increase (CU/CN) may be over 5-times higher than the average normal concentration. Chemically, CMPF is a weak organic base with a mass of 240 Da, and shows a strong lipophilic character. As much as 99.5% of all CMPF may be found bound to HSA in serum. Consequently, this high association to HSA may prevent a sufficient secretion due to lower renal clearance rates in uremic patients, for example, at a rate of about 0.05 mL/min, compared to 0.40 mL/min for healthy patients. Due to its strong binding affinity to HSA, the removal of CMPF through conventional hemodialysis is generally difficult and even practically ineffective.

Accordingly, treatment processes according to some embodiments may use or include an adsorptive device configured to have an effective depletion ability resulting in a normal physiological range. In some embodiments, an effective depletion per treatment may be up to 500 mg/L. Some embodiments may include various approaches leading to the reduction (or even elimination) of CMPF from patients' sera. In various embodiments, treatment processes may be configured to remove CMPF using fractionated plasma separation and adsorption (FPSA) alone or in combination with dialysis. In other embodiments, treatment processes may be configured to remove CMPF using a therapeutic apheresis approach with an exogenous binding competitor (“displacer”). In various embodiments, the displacer may be configured to target compounds that bind CMPF, for instance, targeting HSA (for example, HSA-binding site 1) with high affinity, or combinations thereof. Embodiments are not so limited, as other approaches may be used.

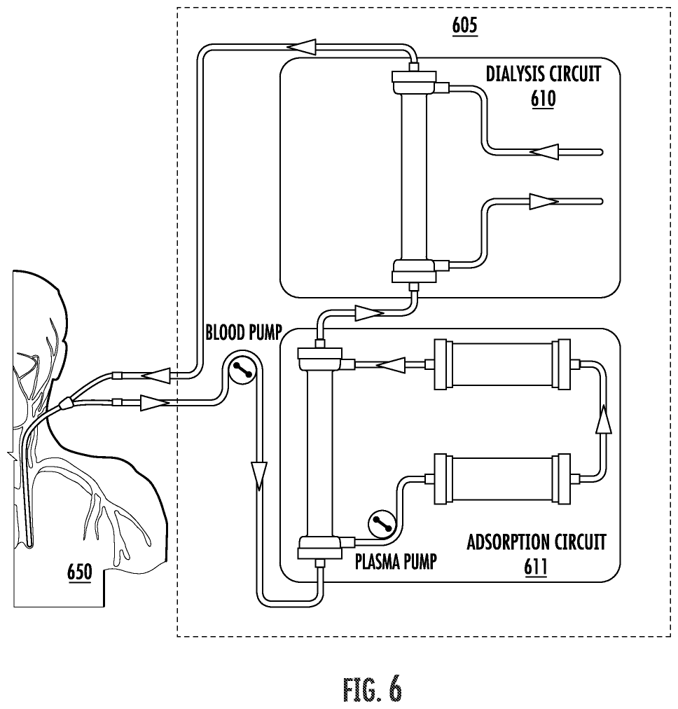

FIG. 6 illustrates a first exemplary Piezo1 agonist removal system in accordance with the present disclosure. As shown in FIG. 6, a patient 650 may be fluidically coupled to a Piezo1 agonist removal system 605. In some embodiments, Piezo1 agonist removal system 605 may include a dialysis circuit or system 610 fluidically coupled to an adsorption circuit or system 611. In various embodiments, adsorption circuit 611 may be or may include an FPSA circuit. In various embodiments, adsorption circuit 611 may perform apheresis during the adsorption process. A non-limiting example of an FPSA circuit may be or may include a Prometheus® machine with FPSA circuit provided by Fresenius Medical Care of Bad Homburg, Germany. In some embodiments, dialysis circuit 610 may be or may include a hemodialysis (HD) system. A non-limiting example of a dialysis circuit 610 may include a Fresenius Polysulfone® high-flux dialyzer provided by Fresenius Medical Care.

In various embodiments, to achieve specific adsorption of CMPF within adsorption circuit 611, a ligand with an affinity towards CMPF may be used. The ligand may be in a competitive range with HSA (for example, a range of about K₁=10⁶ to 10⁸; see, for example, Sakai et al., “Characterization of binding site of uremic toxins on human serum albumin,” Biol Pharm Bull 18(12):1755-61 (1995) and Hendersen et al., “Interaction of 3-carboxy-4-methyl-5-propyl-2-furanpropanoic acid, an inhibitor of plasma protein binding in uraemia, with human albumin,” Biochem Pharmacol 40(11):2543-48 (1990), both of which are incorporated by reference as if fully set forth herein). In addition, in exemplary embodiments, the binding affinity of the CMPF-specific adsorber-ligand towards other HSA-binding site 1-binders, like bilirubin, may be low.

Structural information on the binding pocket of HSA reveals that the main interactions with CMPF are between the residues Tyr-150, Lys-199, Arg-222 and Arg-257 with bond lengths of 2.9 A, 3.0 A, 3.0 A and 3.2 A respectively (see, for example, Faiza 2017). Transferring/conserving this binding-pocket through molecular imprints obtained from CMPF-interaction may be used to design/develop a binding site-mimic with the appropriate pocket-size and physico-chemical force-densities targeting mainly CMPF.

Accordingly, adsorption circuit 611 may be configured to remove CMPF from patients' serum via a fractionated plasma separation and adsorption (FPSA) process combined with conventional hemodialysis (HD). Various sorbent materials for CMPF may be used. In some embodiments, a sorbent material may be any material having an affinity for CMPF sufficient to remove CMPF from the serum as it travels through adsorption circuit 611. In one example, a CMPF-ligand may exhibit binding affinities in the same molar-regime as albumin towards CMPF.

In a first step, an albumin-rich plasma-fraction may be separated and brought in contact with a sorbent material carrying a CMPF-specific ligand to remove free and albumin-bound CMPF as the patient blood travels through adsorption circuit 611. The purified plasma may then reunite with the bloodstream, which is forwarded to a conventional HD step performed via dialysis circuit 610.

In some embodiments, instead of relying on a specific ligand to deplete CMPF from the plasma-stream or using albumin, a displacer compound having a higher binding affinity towards albumin (for instance, binding site 1) may be presented to the albumin-enriched plasma. One non-limiting compound (or displacer) may be or may include dithymoquinone (DTQ) or chemical analogues thereof. DTQ exhibits a higher binding affinity towards HSA's binding site 1 than CMPF, which may free-up CMPF and, thus make it available to be adsorbed by sorbent material (see, for example, Faiza et al., “Dithymoquinone as a novel inhibitor for 3-carboxy-4-methyl-5-propyl-2-furanpropanoic acid (CMPF) to prevent renal failure,” Quantitative Methods, Jul. 23, 2017, which is incorporated by reference as if fully set forth in the present disclosure (“Faiza 2017”). Although DTQ or chemical analogues are used in examples, embodiments are not so limited, as any type of displacer capable of operating according to some embodiments (for instance, ibuprofen) is contemplated in the present disclosure.

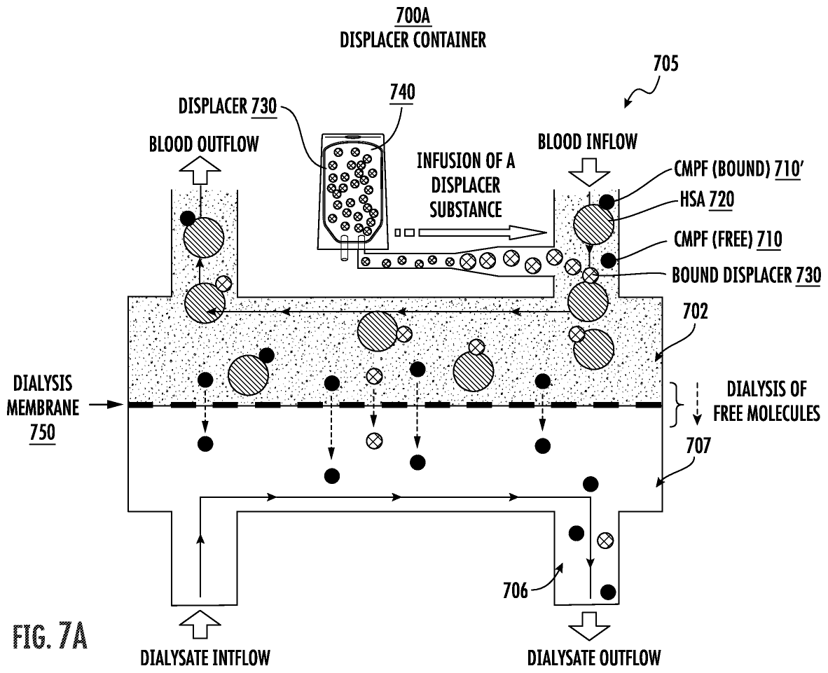

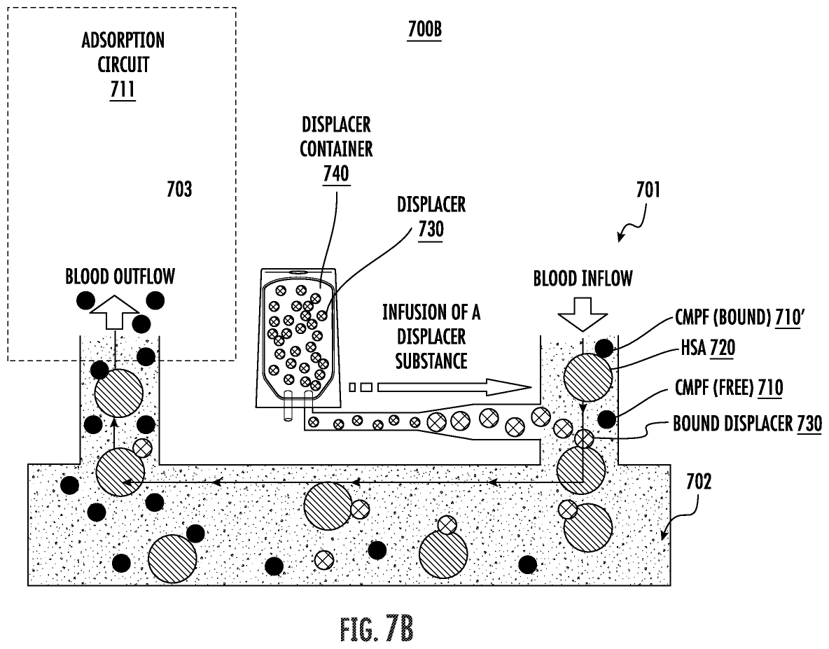

FIG. 7A illustrates a second exemplary Piezo1 agonist removal system in accordance with the present disclosure. More specifically, FIG. 7A depicts an example of dialysis clearance of CMPF using a displacer according to some embodiments of the present disclosure. As depicted in FIG. 7A, a dialysis machine 705 may operate to cause a dialysate inflow of a dialysis fluid 704 and a dialysis outflow of the dialysate fluid along with unwanted substances 706. Patient blood 702 may include a target substance in the form of CMPF 710′ bound to albumin 720 and free or unbound CMPF 710. Unbound CMPF 710 may cross a dialysis membrane 750 and be removed as an unwanted substance 706 with the dialysate outflow. Bound CMPF 710′ is not able to cross dialysis membrane 750 and, therefore, cannot be removed as an unwanted substance 706 with the dialysate outflow.

In some embodiments, dialysis machine 705 may include or may be in fluid communication with a displacer container 740 operative to facilitate the infusion of a displacer 730 into patient blood 702 via a patient blood inflow. As shown in FIG. 7A, displacer 730 may compete for binding sites on HSA 720, leading to a decrease (or even an elimination) of bound CMPF 710 and an increase in free CMPF 710. An increase in free CMPF 710 may facilitate the removal of, or removal of a greater amount of, CMPF 710 from patient blood 702 than could be achieved in the absence of displacer 730.

FIG. 7B illustrates a third exemplary Piezo1 agonist removal system in accordance with the present disclosure. In the system of FIG. 7B, blood outflow 703 with increased free CMPF due to the displacement process may flow into an adsorption circuit 711 configured according to some embodiments. Displacer 730, such as DTQ, exhibits a higher binding affinity towards HSA's binding site 1 than CMPF, which may free-up CMPF and, thus make it available to be adsorbed by sorbent material within adsorption circuit 711. In some embodiments, adsorption circuit 711 may be fluidically coupled to a dialysis system (not shown; see, for example, FIG. 6).

In some embodiments, blood inflow 701 may be from an apheresis process (for instance, blood inflow 701 is actually “plasma inflow” and blood outflow 703 is actually “plasma outflow”). In such an embodiments, “plasma outflow” 703 may reunite the plasma with the red blood cells after the displacement circuit.

Accordingly, referring to FIGS. 7A and 7B, displacer-based treatment processes may provide various pathways to remove CMPF from blood: (1) through a displacer process, then dialysis; (2) through a displacer process, then through an adsorption circuit, (3) through a displacer process within an adsorption circuit, and/or (4) through a displacer process, through an adsorption circuit, then dialysis. In reference to FIG. 6, pathway (3) may include using a displacer within adsorption circuit 611, for instance, within an adsorption filter module. Embodiments are not limited in this context.

Although the displacer process is shown used in combination with HD and/or the adsorption process, embodiments are not so limited. For example, the displacer method may be used without using the adsorption process. In various embodiments, for instance, the displacer method may be used in combination with HD, hemofiltration, hemodiafiltration without using the adsorption process.

In some embodiments, RBC characteristics of the patient RBC population may be determined using various models. For example, an average RBC lifespan of the patient by determining a RBC lifespan using a mathematical model of erythropoiesis. The RBC characteristics may be determined before, during, and/or after treatment using treatment processes according to some embodiments to increase RBC lifespan and, therefore, the RBC population of the patient. In this manner, a healthcare professional may use the RBC characteristics to determine configurations of the treatment processes and/or to determine progress (for instance, an increase in RBC lifespan as a result to treatment processes according to some embodiments).

Non-limiting examples of RBC biological models that may be used to determine RBC characteristics may include Fuertinger et al., “A model of erythropoiesis in adults with sufficient iron availability,” J Math Biol. 2013 May; 66(6):1209-40 and Fuertinger et al., and “Prediction of hemoglobin levels in individual hemodialysis patients by means of a mathematical model of erythropoiesis,” PLOS ONE, Apr. 18, 2018, both of which are incorporated by reference as if fully set forth herein.

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A method of treating a patient with renal anemia, comprising:
increasing a red blood cell (RBC) lifespan of an RBC population of the patient via reduction of a Piezo1 channel activation duration of at least a portion of the RBC population by reducing an amount of a target uremic compound in the blood of the patient, the target uremic compound having a form that prolongs the Piezo1 channel activation duration,
wherein the amount of the target uremic compound is reduced via selectively removing at least a portion of the target uremic compound from the blood of the patient.

2. The method of claim 1, wherein the target uremic compound is 3-carboxy-4-methyl-5-propyl-2-furanpropionate (CMPF).

3. The method of claim 1, further comprising monitoring an average RBC lifespan of the using a mathematical model of erythropoiesis.

4. The method of claim 1, wherein the patient is receiving a maximum dosage of at least one erythropoietin stimulating agents (ESA) to treat renal anemia.

5. The method of claim 4, further comprising reducing the ESA dosage based on an increase in the RBC lifespan of the patient.

6. The method of claim 1, wherein selectively removing the target uremic compound includes an adsorption process performed on blood of the patient.

7. The method of claim 6, wherein the adsorption process comprises fractionated plasma separation and adsorption (FPSA).

8. The method of claim 6, the adsorption process using a ligand to adsorb CMPF, the ligand having a binding affinity to CMPF in the range of about K₁=10⁶ to 10⁸.

9. The method of claim 1, comprising performing apheresis to selectively remove the target uremic compound.

10. The method of claim 8, comprising performing apheresis with a displacer targeting an RBC binding site of the at least one targeted uremic compound.

11. The method of claim 9, the displacer comprising dithymoquinone (DTQ) or chemical analogues thereof.

12. An apparatus for treating a patient with renal anemia, the apparatus comprising:
a target compound reduction system configured to engage blood of the patient to reduce an amount of a target compound from the blood of the patient by selectively removing at least a portion of the target compound from the blood of the patient,
wherein reducing the amount of the target compound in the blood of the patient increases a red blood cell (RBC) lifespan of an RBC population of blood of the patient via reduction of a Piezo1 channel activation duration of at least a portion of the RBC population, the target compound having a form that prolongs the Piezo1 channel activation duration.

13. The apparatus of claim 12, wherein the target compound is a uremic compound.

14. The apparatus of claim 12, wherein the target compound is 3-carboxy-4-methyl-5-propyl-2-furanpropionate (CMPF).

15. The apparatus of claim 12, the target compound reduction system operative to perform an adsorption process on the blood of the patient.

16. The apparatus of claim 15, wherein the adsorption process comprises fractionated plasma separation and adsorption (FPSA).

17. The apparatus of claim 16, wherein the adsorption process uses a ligand to adsorb CMPF, the ligand having a binding affinity to CMPF in the range of about K₁=10⁶ to 10⁸.

18. The apparatus of claim 12, the target compound reduction system operative to perform apheresis to selectively remove the target compound.

19. The apparatus of claim 18, wherein apheresis is performed with a displacer targeting a RBC binding site of the at least one target compound.

20. The apparatus of claim 19, the displacer comprising dithymoquinone (DTQ) or chemical analogues thereof.
